SEQUENCE LISTING (24 sequences)

SEQ ID NO: 1 - DNA, 1052 bp, Homo sapiens
ggtaccatgaagtgggtggaatcaatttttttaattttcctactaaattttactgaatccagaacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaagggaattc

SEQ ID NO: 2 - DNA, 1466 bp, Homo sapiens
ggtaccatgaagtgggtggaatcaatttttttaattttcctactaaattttactgaatccagaacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttagggaattc

SEQ ID NO: 3 - DNA, 1010 bp, Homo sapiens
ggtaccatggccgggaccgtgcgcaccgcgtgcttggtggtggcgatgctgctcagcttggacttcccgggacaggcgcagcccccgccgccgccgccggacgccacctgtcaccaagtccgctccttcttccagagactgcagcccggactcaagtgggtgccagaaactcccgtgccaggatcagatttgcaagtatgtctccctaagggcccaacatgctgctcaagaaagatggaagaaaaataccaactaacagcacgattgaacatggaacagctgcttcagtctgcaagtatggagctcaagttcttaattattcagaatgctgcggttttccaagaggcctttgaaattgttgttcgccatgccaagaactacaccaatgccatgttcaagaacaactacccaagcctgactccacaagcttttgagtttgtgggtgaatttttcacagatgtgtctctctacatcttgggttctgacatcaatgtagatgacatggtcaatgaattgtttgacagcctgtttccagtcatctatacccagctaatgaacccaggcctgcctgattcagccttggacatcaatgagtgcctccgaggagcaagacgtgacctgaaagtatttgggaatttccccaagcttattatgacccaggtttccaagtcactgcaagtcactaggatcttccttcaggctctgaatcttggaattgaagtgatcaacacaactgatcacctgaagttcagtaaggactgtggccgaatgctcaccagaatgtggtactgctcttactgccagggactgatgatggttaaaccctgtggcggttactgcaatgtggtcatgcaaggctgtatggcaggtgtggtggagattgacaagtactggagagaatacattctgtcccttgaagaacttgtgaatggcatgtacagaatctatgacatggagaacgtactgcttggtctcttttcaacaatccatgattctatccaggggaattc

SEQ ID NO: 4 - DNA, 992 bp, Homo sapiens
ggtaccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaagggaattc

SEQ ID NO: 5 - DNA, 554 bp, Homo sapiens
ggtaccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagttggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgcgggaattc

SEQ ID NO: 6 - DNA, 941 bp, Homo sapiens
ggtaccatgtctcttgagcagaggagtctgcactgcaagcctgaggaagcccttgaggcccaacaagaggccctgggcctggtgtgtgtgcaggctgccgcctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggcccttgctgaaaccagctatgtgaaagtccttgagtatgtgatcaaggtcagtgcaagagttcgctttttcttcccatccctgcgtgaagcagctttgagagaggaggaagagggagtcgggaattc

SEQ ID NO: 7 - DNA, 996 bp, Homo sapiens
ggtaccacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaataagaattc

SEQ ID NO: 8 - DNA, 1410 bp, Homo sapiens
  misc_feature (907)..(907): n is a, c, g, or t
ggtaccacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttataagaattc

SEQ ID NO: 9 - DNA, 921 bp, Homo sapiens
ggtaccccggacgccacctgtcaccaagtccgctccttcttccagagactgcagcccggactcaagtgggtgccagaaactcccgtgccaggatcagatttgcaagtatgtctccctaagggcccaacatgctgctcaagaaagatggaagaaaaataccaactaacagcacgattgaacatggaacagctgcttcagtctgcaagtatggagctcaagttcttaattattcagaatgctgcggttttccaagaggcctttgaaattgttgttcgccatgccaagaactacaccaatgccatgttcaagaacaactacccaagcctgactccacaagcttttgagtttgtgggtgaatttttcacagatgtgtctctctacatcttgggttctgacatcaatgtagatgacatggtcaatgaattgtttgacagcctgtttccagtcatctatacccagctaatgaacccaggcctgcctgattcagccttggacatcaatgagtgcctccgaggagcaagacgtgacctgaaagtatttgggaatttccccaagcttattatgacccaggtttccaagtcactgcaagtcactaggatcttccttcaggctctgaatcttggaattgaagtgatcaacacaactgatcacctgaagttcagtaaggactgtggccgaatgctcaccagaatgtggtactgctcttactgccagggactgatgatggttaaaccctgtggcggttactgcaatgtggtcatgcaaggctgtatggcaggtgtggtggagattgacaagtactggagagaatacattctgtcccttgaagaacttgtgaatggcatgtacagaatctatgacatggagaacgtactgcttggtctcttttcaacaatccatgattctatccagtgagaattc

SEQ ID NO: 10 - DNA, 990 bp, Homo sapiens
ggtaccgaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatgagaattc

SEQ ID NO: 11 - DNA, 555 bp, Homo sapiens
ggtaccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagttggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagaattc

SEQ ID NO: 12 - DNA, 939 bp, Homo sapiens
ggtacctctcttgagcagaggagtctgcactgcaagcctgaggaagcccttgaggcccaacaagaggccctgggcctggtgtgtgtgcaggctgccgcctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggcccttgctgaaaccagctatgtgaaagtccttgagtatgtgatcaaggtcagtgcaagagttcgctttttcttcccatccctgcgtgaagcagctttgagagaggaggaagagggagtctgagaattc

SEQ ID NO: 13 - protein, 346 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGE

SEQ ID NO: 14 - protein, 484 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHL

SEQ ID NO: 15 - protein, 332 aa, Homo sapiens
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQ

SEQ ID NO: 16 - protein, 326 aa, Homo sapiens
MEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGE

SEQ ID NO: 17 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 18 - protein, 309 aa, Homo sapiens
MSLEQRSLHCKPEEALEAQQEALGLVCVQAAASSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 19 - protein, 327 aa, Homo sapiens
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGE

SEQ ID NO: 20 - protein, 465 aa, Homo sapiens
  misc_feature (301)..(301): Xaa can be any naturally occurring amino acid
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHL

SEQ ID NO: 21 - protein, 303 aa, Homo sapiens
PDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQZ

SEQ ID NO: 22 - protein, 326 aa, Homo sapiens
EEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEZ

SEQ ID NO: 23 - protein, 180 aa, Homo sapiens; identical to SEQ ID NO: 17

SEQ ID NO: 24 - protein, 308 aa, Homo sapiens
SLEQRSLHCKPEEALEAQQEALGLVCVQAAASSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

(19) United States
(12) Patent Application Publication
(10) Pub. No.: US 2010/0235932 A1
(43) Pub. Date: Sep. 16, 2010

(54) ANIMAL MODELS CARRYING TUMORS EXPRESSING HUMAN LIVER CANCER-SPECIFIC ANTIGEN AND METHOD FOR ANALYZING PREVENTION AND TREATMENT EFFICACY OF DENDRITIC CELLS-DERIVED IMMUNOTHERAPEUTICS USING THE ABOVE

(75) Inventors: Yong-Soo Bae, Gyeonggi-do (KR); Hyun-Soo Lee, Seoul (KR); Mi-Kyung Min, Seoul (KR); Cheol-Woong Jeong, Seoul (KR); Kyu-Ho Kang, Seoul (KR)

Correspondence Address: CLARK & ELBING LLP, 101 FEDERAL STREET, BOSTON, MA, 02110, US

(73) Assignee: CREAGENE INC., Sungman-si (KR)

(21) Appl. No.: 12/451,508

(22) PCT Filed: Mar. 13, 2008
(86) PCT No.: PCT/KR2008/001420

§ 371 Date: May 19, 2010

(30) Foreign Application Priority Data
May 17, 2007 (KR) 10-2007-0048213

(51) Int. Cl. A01K 67/00; C12N 5/071

(52) U.S. Cl. 800/10; 435/354

(57) ABSTRACT

The present invention relates to a method for analyzing the prevention and treatment efficacy of a dendritic cell-derived immunotherapeutic for liver cancer using an animal model carrying tumors expressing a human liver cancer-specific antigen, which comprises the steps of: (a) (a1) administering to a normal animal other than human dendritic cells to be analyzed, or (a1) administering to a normal animal other than human a cancer cell line expressing the human liver cancer-specific antigen to induce cancer in the normal animal; (b) (b1) administering to the animal the cancer cell line expressing the human liver cancer-specific antigen to induce cancer in the animal when the step (a1) is performed in the step (a), or (b1) administering to the animal with cancer dendritic cells to be analyzed when the step (a1) is performed in the step (a); and (c) determining the prevention and treatment efficacy of the dendritic cells as immunotherapeutics for liver cancer by measuring the formation or growth of cancer cells originated from the cancer cell line in the animal.

### BACKGROUND OF THE INVENTION

1. Field of the Invention

The present invention relates to a method for analyzing the prevention and treatment efficacy of a dendritic cell-derived immunotherapeutic for liver cancer, more particularly, to a method for a method for analyzing the prevention and treatment efficacy of a dendritic cell-derived immunotherapeutic for liver cancer using an animal model bearing human liver cancer.

2. Description of the Related Art

The annual incidence rate of the liver cancer is 4% of whole cancer which corresponds to 560 thousands patients and 390 thousands patients reside in Asia. In South Korea, 12-15 thousands of new patients suffering from liver cancer occur every year, which is second ranked incidence rate following stomach cancer. The significance of the liver cancer cannot be overlook because of the second highest mortality behind lung cancer and stomach cancer. 70% of liver cancer is caused by the infection of hepatitis B virus, 13% of liver cancer is estimated to be caused by hepatitis C virus, and the other causes occupy 18% of the liver cancer incidence.

While the liver cancer is classified as primary cancer and metastatic cancer, the most frequently occurred primary liver cancer is hepatocellular carcinoma (HCC) and the incidence rate of metastatic liver cancer that spread through hepatic portal vein is also high.

Much effort to develop therapeutics for liver diseases is mainly focused on the fields of hepatitis. Hepatitis therapeutics such as Interferon and Lamivudine have been developed and used currently. Lamivudine has little side effects compared to Interferon and is easily administered via oral route. However, it is reported that the occurrence rate of viruses having tolerances to Lamivudine come up to almost 50% and it exerts little therapeutic effects to the patients who have been entered into liver cancer.

The patient having liver cancer in the early stage is asymptomatic and the conditions of the patient have already become danger after being diagnosed as cancer. Even though the primary therapeutic method for liver cancer is surgical excision, the number of patients who are capable of being taken surgical operation is very small. The examples of other therapeutic methods for liver cancer comprise tissue transplantation, systemic chemotheraphy, radiotheraphy, and fulguration. However, these methods represent high rate of relapse and raise severe side effects such as transplantation rejection. Even successful excision operations have annual relapse rate of 25%. It is also known that the successful results of the surgical operation are obtained in the patient who has small tumor size of 2-3 cm. However, the possibility of relapse within three years after operation even in the small tumor size liver cancer is estimated to over 50%. The high relapse of liver cancer is caused firstly by micrometastasis during the excision operation and secondly by occurrence of new liver cancer from cirrhosis.

There remains a need for the novel cellular immunotherapy for liver cancer which has little side effects and pains. Cancer vaccination using dendritic cells has been currently developed and known as active immunotherapy. It has stronger effect compared to the immunization with killed cancer cell, longer effect than passive immunotherapy which injects in vitro cultured patient's T cell, and improved safety compared to directly administering cytokines such as IL-2 and IFN-α. In addition, it has remarkable therapeutic effectiveness for metastatic or recurrent cancer and has little side effects and pains. Although it is difficult to reduce the size of tumor using cancer vaccination with dendritic cells, however, by inducing immune responses in the body, it exerts significant effect of inhibiting relapse or overt metastasis in the early stage of metastasis such as micrometastasis or in the post-primary therapy stage.

For clinical tests of immunotherapy using dendritic cells, it is prerequisite to examine their efficacy and safety in animal models. However, there has not been yet proposed prostate cancer animal models for evaluating dendritic cell-based vaccines against human prostate cancer.

Throughout this application, various patents and publications are referenced and citations are provided in parentheses. The disclosure of these patents and publications in their entities are hereby incorporated by references into this application in order to more fully describe this invention and the state of the art to which this invention pertains.

### DETAILED DESCRIPTION OF THIS INVENTION

Endeavoring to meet the need in the art described above, the present inventors have established xenogenic cancer cell lines expressing human liver cancer-specific antigens and animal models using them. In addition, we have found that the prevention and treatment efficacy of dendritic cells as immunotherapeutics for liver cancer could be accurately analyzed using the animal models.

Accordingly, it is an object of this invention to provide a method for analyzing the prevention and treatment efficacy of a dendritic cell-derived immunotherapeutic for liver cancer.

It is another object of this invention to provide a mouse-derived liver cancer cell line expressing a human liver cancer-specific antigen.

It is still another object to this invention to provide a mouse (Mus musculus) liver cancer model.

Other objects and advantages of the present invention will become apparent from the following detailed description together with the appended claims and drawings.

In one aspect of this invention, there is provided a method for analyzing the prevention and treatment efficacy of a dendritic cell-derived immunotherapeutic for liver cancer using an animal model carrying tumors expressing a human liver cancer-specific antigen, which comprises the steps of: (a) (a′) administering to a normal animal other than human dendritic cells to be analyzed, or (a″) administering to a normal animal other than human a cancer cell line expressing the human liver cancer-specific antigen to induce cancer in the normal animal; (b) (b′) administering to the animal the cancer cell line expressing the human liver cancer-specific antigen to induce cancer in the animal when the step (a′) is performed in the step (a), or (b″) administering to the animal with cancer dendritic cells to be analyzed when the step (a″) is performed in the step (a); and (c) determining the prevention and treatment efficacy of the dendritic cells as immunotherapeutics for liver cancer by measuring the formation or growth of cancer cells originated from the cancer cell line in the animal.

The present invention is directed to (i) methods for analyzing the prevention efficacy of dendritic cell-derived immunotherapeutic for liver cancer and (ii) methods for analyzing the treatment efficacy of a dendritic cell-derived immunotherapeutic for liver cancer.

In this regard, the present method for analyzing the treatment efficacy of a dendritic cell-derived immunotherapeutic for liver cancer comprises the steps of (a″) administering to a normal animal other than human a cancer cell line expressing the human liver cancer-specific antigen to induce cancer in the normal animal; (b″) administering to the animal with cancer dendritic cells to be analyzed; and (c) determining the treatment efficacy of the dendritic cells as immunotherapeutics for liver cancer by measuring the formation or growth of cancer cells originated from the cancer cell line in the animal.

The present method for analyzing the prevention efficacy of a dendritic cell-derived immunotherapeutic for liver cancer comprises the steps of (a′) administering to a normal animal other than human dendritic cells to be analyzed; (b′) administering to the animal the cancer cell line expressing the human liver cancer-specific antigen to induce cancer in the animal; and (c) determining the prevention of the dendritic cells as immunotherapeutics for liver cancer by measuring the formation or growth of cancer cells originated from the cancer cell line in the animal.

The present invention provides firstly a successful protocol for analyzing the efficacy of a human dendritic cell-derived iramunotherapeutic for liver cancer using animal models. According to conventional technologies, animal models have not yet been provided for such analysis.

In the present invention, animals used include any animal species except for human, preferably mammals, more preferably rodents, still more preferably a mouse (Mus musculus), and most preferably C3H/HeN mouse. The term used herein “normal animal” refers to animals having not cancer.

According to the present method, an antigen used to establish a cancer cell line expressing a human liver cancer-specific antigen includes any antigen expressed in human liver cancer cells. Preferably, the human liver cancer-specific antigen is AFP (Alpha-Fetoprotein), GPC3 (Glypican3), TRP53 (Transformation Related Protein 53), MAGEA 1 (Melanoma Antigen Family A, 1), NY-ESO-1 (New York Esophageal Squamous Cell Carcinoma 1 or Cancer/Testis Antigen1; CTAG1), more preferably AFP (Alpha-Fetoprotein), GPC3 (Glypican3), TRP53 (Transformation Related Protein 53) or MAGEA 1 (Melanoma Antigen Family A, 1), still more preferably AFP (Alpha-Fetoprotein) or GPC3 (Glypican3), most preferably AFP (Alpha-Fetoprotein). The human liver cancer-specific antigens may comprise natural-occurring full length amino acid sequences as well as their partial sequences.

Preferably, the antigen useful in this invention comprises an amino acid sequence spanning amino acids 1-346 or 1-484 of SEQ ID NO: 13 or 14 for AFP (Alpha-Fetoprotein), an amino acid sequence spanning amino acids 1-332 of SEQ ID NO: 15 for GPC3 (Glypican3), an amino acid sequence spanning amino acids 1-326 of SEQ ID NO: 16 for TRP53 (Transformation Related Protein 53), an amino acid sequence spanning amino acids 1-180 of SEQ ID NO: 17 for NY-ESO-1 (New York Esophageal Squamous Cell Carcinoma 1 or Cancer/Testis Antigen1; CTAG1), or an amino acid sequence spanning amino acids 1-309 of SEQ ID NO: 18 for MAGEA 1 (Melanoma Antigen Family A, 1).

The cancer cell lines used in cancer induction in normal animals may be derived from various animals. Preferably, the cancer cell line is allogeneic or syngeneic to the recipient animal, more preferably syngeneic to the recipient animal. According to a preferred embodiment, a mouse is used as normal animals and a mouse-derived cancer cell line is used as cancer cell lines. More preferably, C3H/HeN mouse is used as normal animals and MH134 mouse-derived cancer cell line is used as cancer cell lines.

The cancer cell lines useful in this invention include liver cancer cell lines, gastric cancer cell lines, brain cancer cell lines, lung cancer cell lines, breast cancer cell lines, ovary cancer cell lines, bronchial cancer cell lines, nasopharyngeal cancer cell lines, laryngeal cancer cell lines, pancreatic cancer cell lines, bladder cancer cell lines, colon cancer cell lines and cervical cancer cell lines. A syngeneic liver cancer cell line, for example MH134 cancer cell line, is the most suitable in this invention. According to a preferred embodiment, cancer cell lines expressing human liver cancer specific antigen used in this invention are derived from liver cancer cell. Meanwhile, there are mouse derived liver cancer cell lines such as C57BL/6 mouse, C3H/HeN mouse, and BALB/c mouse-derived liver cancer cell lines, however, they are not suitable to be used directly in this invention since they do not express the above-mentioned liver cancer specific antigen. Where liver cancer cell lines are used as cancer cell lines and C3H/HeN mice are used as recipient animals, a syngeneic liver cancer cell line, MH134, is the most suitable in this invention.

Mouse-derived liver cancer cell lines are transformed with nucleotide sequences encoding human liver-specific antigens and then used in the present invention. Human liver cancer-specific antigen-encoding nucleotide sequences may comprise natural-occurring full length nucleotide sequences as well as their partial sequences. Preferably, the nucleotide sequence encoding human liver cancer-specific antigens useful in this invention comprises a nucleotide sequence encoding an amino acid sequence spanning amino acids 1-346 or 1-484 of AFP (Alpha-Fetoprotein), an amino acid sequence spanning amino acids 1-332 of GPC3 (Glypican3), an amino acid sequence spanning amino acids 1-326 of TRP53 (Transformation Related Protein 53), an amino acid sequence spanning amino acids 1-180 of NY-ESO-1 (New York Esophageal Squamous Cell Carcinoma 1 or Cancer/Testis Antigen1; CTAG1), or an amino acid sequence spanning amino acids 1-309 of MAGEA1 (Melanoma Antigen Family A, 1). More preferably, the nucleotide sequence encoding human liver cancer-specific antigens comprises a nucleotide sequence of nucleotides 7-1044 of SEQ ID NO: 1 or nucleotides 7-1458 of SEQ ID NO: 2 for AFP (Alpha-Fetoprotein), a nucleotide sequence of nucleotides 7-1002 of SEQ ID NO: 3 for GPC3 (Glypican3), a nucleotide sequence of nucleotides 7-984 of SEQ ID NO: 4 for TRP53 (Transformation Related Protein 53), a nucleotide sequence of nucleotides 7-546 of SEQ ID NO: 5 for NY-ESO-1 (New York Esophageal Squamous Cell Carcinoma 1 or Cancer/Testis Antigen1; CTAG1), or a nucleotide sequence of nucleotides 7-933 of SEQ ID NO: 6 for MAGEA1 (Melanoma Antigen Family A, 1).

The nucleotide sequences coding for human liver cancer-specific antigens may be prepared by a variety of methods. For instance, total RNA is isolated from human-derived liver cancer cell line (e.g., HepG2, ZR75-1, SK-BR-3), from which cDNA molecules are synthesized using primers designed by referring to known nucleotide sequences encoding human liver cancer-specific antigens. The cDNA molecules synthesized thus are cloned into suitable expression vectors for animal cells (e.g., pcDNA3.1 (+)) and introduced into mouse liver cancer cells (e.g., MH134 cell line). Among cells, transformed cancer cells expressing human liver cancer-specific antigens are selected and used to establish cancer cell line expressing human liver cancer-specific antigens. As described above, human liver cancer-specific, antigens-expressing mouse derived liver cancer cell lines are established for the first time by the present inventors. The cancer cell line expressing a human liver cancer-specific antigen processes the human liver cancer-specific antigen expressed and displays the processed antigen molecule on its surface through Major Histocompatibility Complex I. As results, the cancer cell line permits to be recognized by T cells specific to the human liver cancer antigen.

Dendritic cells to be analyzed in this invention may be prepared by various protocols known to one of skill in the art. For example, dendritic cells are obtained from monocytes, hematopoietic progenitor cells or bone marrow cells.

The preparation process for dendritic cells using bone marrow cells are exemplified as follows: Bone marrow cells are isolated from a femur and tibia of mice and the cultured in media containing suitable cytokines (e.g., IL-4 and GM-CSF) for the differentiation to dendritic cells. The immature dendritic cells obtained thus are pulsed with a human liver cancer-specific antigen and then cultured in media containing suitable cytokines for maturating dendritic cells, which serve as samples to be analyzed. The pulsing becomes very effective when CTP (cytoplasmic transduction peptide)-conjugated antigens are used. The CTP molecule delivers antigens into cytoplasm not nucleus, which permits dendritic cells to present more effectively antigens on their surface through Major Histocompatibility Complex Class I (MHC I) molecules. The descriptions of CTP molecules are also found in Korean Patent No 10-0608558, the teachings of which are incorporated herein by reference.

The dendritic cells to be analyzed may be administered into animals via various routes, preferably intravenous injection or subcutaneous injection, most preferably subcutaneous injection. The cancer cell lines expressing human liver cancer-specific antigens may be administered into normal animals via various routes, preferably intravenous injection or subcutaneous injection, most preferably subcutaneous injection (Fong, L. et al., Dendritic cells injected via different routes induce immunity in cancer patients. J. Immunol. 166:4254. (2001)).

The dendritic cells in the step (a) are administered into animals, e.g. mice in a dose of 1×10⁴-1×10⁸ cells, preferably 1×10⁵-1×10⁷ cells and more preferably about 1×10⁶ cells. It is preferred that the administration of dendritic cells is performed twice in a suitable time interval (e.g., one week). The cancer cell line in the step (a) are administered into animals, e.g. mice in a dose of 1×10⁴-1×10⁸ cells, preferably 1×10⁵-1×10⁷ cells and more preferably about 3×10⁵ cells.

Based on knowledge available to one of skill in the art, it could be generally believed that when cancer cell lines expressing human liver cancer-specific antigens are administered to animal except for human, they are very likely to be eliminated by immune reactions in animals. Surprisingly, according to the present invention, human liver cancer specific antigen-expressing cancer cell lines administered to animal except for human induce the formation of cancerous tissues in animals, which enables the present method to be successfully performed.

The administration route and dose of cancer cell lines in the step (a) described above can be also applied to the step (b).

According to the present invention, (i) the human liver cancer-specific antigen used to pulse dendritic cells in the step (a′) and (ii) the human liver cancer-specific antigen expressed in the cancer cell line of the step (b′) are originated from the same one antigen. For instance, where the human liver cancer-specific antigen used to pulse dendritic cells in the step (a′) is AFP (Alpha-Fetoprotein), the human liver cancer-specific antigen expressed in the cancer cell line of the step (b′) expresses is also AFP. Therefore, cytotoxic T lymphocytes induced by dendritic cells presenting AFP (Alpha-Fetoprotein) recognize cancer cell lines expressing AFP, resulting in the lysis of cancer cell lines.

According to the present invention, (i) the human liver cancer-specific antigen used to pulse dendritic cells in the step (a″) and (ii) the human liver cancer-specific antigen expressed in the cancer cell line of the step (b″) are originated from the same one antigen. For instance, where the human liver cancer-specific antigen used to pulse dendritic cells in the step (a″) is AFP, the human liver cancer-specific antigen expressed in the cancer cell line of the step (b′) expresses is also AFP (Alpha-Fetoprotein). Therefore, cytotoxic T lymphocytes induced by dendritic cells presenting AFP (Alpha-Fetoprotein) recognize cancer cell lines expressing AFP, resulting in the lysis of cancer cell lines.

In the final step of the present invention, the formation or growth of cancer cells in animals are measured to determine the prevention or treatment efficacy of the dendritic cells as immunotherapeutics for liver cancer. The formation or growth of cancer cells in animals can be evaluated with naked eye or using devices such as calipers. Where the further formation or growth of cancer cells are observed, it can be determined that dendritic cells of interest as immunotherapeutics possess the prevention or treatment efficacy for liver cancer.

For executing the prevention or treatment of liver cancer using dendritic cells in a clinical scale, it is prerequisite to verity the efficacy and safety of dendritic cells in animal models. The present invention allows for animal model-based evaluation of dendritic cells as immunotherapeutics. Dendritic cells selected by the present invention become promising candidates as immunotherapeutics for liver cancer.

In another aspect of this invention, there is provided a mouse-derived liver cancer cell line (recombinant MH134 cell line) expressing a human liver cancer-specific antigen, characterized in that the human liver cancer-specific antigen is AFP (Alpha-Fetoprotein), GPC3 (Glypican3), TRP53 (Transformation Related Protein 53), MAGEA1 (Melanoma Antigen Family A, 1), or NY-ESO-1 (New York Esophageal Squamous Cell Carcinoma 1 or Cancer/Testis Antigen1; CTAG1).

The mouse-derived liver cancer cell line (recombinant MH134 cell line) expressing a human liver cancer-specific antigen of this invention has been firstly developed by the present inventors for establishing liver cancer animal models.

The liver cancer cell line of this invention is prepared by transforming with a nucleotide sequence encoding AFP (Alpha-Fetoprotein), GPC3 (Glypican3), TRP53 (Transformation Related Protein 53), MAGEA1 (Melanoma Antigen Family A, 1), or NY-ESO-1 (New York Esophageal Squamous Cell Carcinoma 1 or Cancer/Testis Antigen1; CTAG1). Human liver cancer-specific antigen-encoding nucleotide sequences may comprise natural-occurring full length nucleotide sequences as well as their partial sequences. Preferably, the liver cancer cell line is transformed with a vector containing a nucleotide sequence encoding an amino acid sequence spanning amino acids 1-346 or 1-484 of AFP, an amino acid sequence spanning amino acids 1-332 of GPC3, an amino acid sequence spanning amino acids 1-326 of TRP53, an amino acid sequence spanning amino acids 1-309 of MAGEA1, or an amino acid sequence spanning amino acids 1-180 of NY-ESO-1.

More preferably, the present mouse-derived liver cancer cell lines expressing human liver cancer specific antigen are ones that have been transformed with a vector pcDNA3.1(+)-Tag/AFP (Alpha-Fetoprotein), pcDNA3.1(+)-Tag/GPC3 (Glypican3), pcDNA3.1(+)-Tag/TRP53 (Transformation Related Protein 53), pcDNA3.1(+)-Tag/NY-ESO-1(New York Esophageal Squamous Cell Carcinoma 1 OR Cancer/Testis Antigen1; CTAG1) or pcDNA3.1(+)-Tag/MAGEA1 (Melanoma Antigen Family A, 1), which have been made by incorporating a nucleotide sequence encoding a human liver cancer specific antigen into pcDNA3.1(+)-Tag (pcDNA3.1(+)-36A) (See FIG. 2.).

Human liver cancer antigen AFP, GPC3, TRP53, NY-ESO-1 and MGAEA1 that are incorporated into the vector pcDNA3.1(+)-Tag as depicted in FIG. 2 are represented by 1038 nucleotides of 7-1044 of SEQ ID NO: 1, 1452 nucleotides of 7-1458 of SEQ ID NO: 2, 996 nucleotides of 7-1002 of SEQ ID NO: 3, 978 nucleotides of 7-984 of SEQ ID NO: 4, 540 nucleotides of 7-546 of SEQ ID NO: 5, and 927 nucleotides of 7-993 of SEQ ID NO: 6, respectively.

The cancer cell line expressing human liver cancer-specific antigens processes the human liver cancer-specific antigen expressed and presents the processed antigen molecule on its surface through Major Histocompatibility. Complex I. As results, the cancer cell line permits to be recognized by T lymphocytes specific to the human liver cancer antigen.

In still another aspect of this invention, there is provided a mouse liver cancer model, characterized in that the mouse model has a cancer formed by inoculating the liver cancer cell line of this invention expressing the human liver cancer-specific antigen, and the metastasis or growth of the cancer formed in the mouse model is inhibited by the treatment of dendritic cells pulsed with the human liver cancer-specific antigen.

The mouse liver cancer model bears a cancer formed by inoculating the mouse liver cancer cell line expressing human liver cancer-specific antigen and allows for the evaluation of dendritic cells as immunotherapeutics for liver cancer. Mouse models have not been yet suggested to evaluate the prevention and treatment efficacy for liver cancer.

According to a preferred embodiment, the liver cancer cell line injected into the mouse is syngeneic to the mouse. According to a preferred embodiment, the mouse liver cancer model is used for performing the present method to analyze the prevention and treatment efficacy of dendritic cells for liver cancer described hereinabove. According to a preferred embodiment, the mouse model of this invention is C3H/HeN mouse syngeneic to the injected cancer cell line.

### BRIEF DESCRIPTION OF THE DRAWINGS

FIG. 1 is a gel photograph showing PCR products of nucleotide sequences encoding liver-specific antigens AFP (Alpha-Fetoprotein), MAGEA1 (Melanoma Antigen Family A, 1), TRP53 (Transformation Related Protein 53), GPC3 (Glypican3) and NY-ESO-1 (New York Esophageal Squamous Cell Carcinoma 1 or Cancer/Testis Antigen1; CTAG1). For preparing recombinant antigens, cDNA molecules were synthesized from HepG2 (Human liver cancer cell line), ZR-75-1 (Human breast cancer cell line), SK-BR3 and used for PCR amplification of nucleotide sequences encoding liver-specific antigens AFP, MAGEA1, TRP53, GPC3, and ESO-1. Lanes M, 1, 2, 3, 4, 5 and 6 denote marker, AFP (Alpha-Fetoprotein) 1/2N (1040 bp), AFP (Alpha-Fetoprotein) 2/3N (1454 bp), GPC3 (Glypican3) 1/2N (998 bp), TRP53 (Transformation Related Protein 53) 2/3N (980 bp), NY-ESO-1 (New York Esophageal Squamous Cell Carcinoma 1 or Cancer/Testis Antigen1; CTAG1) (540 bp), MAGEA1 (Melanoma Antigen Family A,1) (929 bp), respectively.

FIG. 2 represents genetic maps and their partial nucleotide sequences of recombinant vectors for expressing liver cancer-specific antigens. Using cDNA molecules synthesized from HepG2 (Human liver cancer cell line), ZR-75-1 (Human breast cancer cell line), Sk-BR3 as templates, nucleotide sequences encoding liver-specific antigens AFP, MAGEA1, TRP53, GPC3 and NY-ESO-1 were amplified by PCR. For expressions, the amplified sequences were cloned into either a eukaryotic vector (pcDNA3.1-36A) or prokaryotic vector (pCTP). In the genetic map of vectors, 36A, CMV promoter, BGH pA, fl ori and SV40 ori represent 36A Tag-encoding sequence, promoter of cytomegalovirus, polyadenylation sequence of bovine growth hormone gene, fl replication origin and SV40 replication origin, respectively. The antibiotics represent antibiotic-resistant genes. 36A Tag-encoding sequence was inserted into the eukaryotic vector in order to facilitate the detection of protein expressed from the vector. Primers for introducing Tag sequence are Tag-XhoI/s(5′-ACCCTCGAGGTCCATGACCGGAGGTCAGC AGATGGGTCGCGACCTGTACGACGA-3′) and Tag-XbaI/as (5′-ACCTCTAGATTAGCTTCCCCATCTGTCCTGTCGTCATCGTCGTACAGGTCGCG-S′). Tag DNA fragments were prepared by PCR amplification under the thermal conditions: 1 cycle of 30 sec at 94° C., 30 sec at 52° C., and 5 min at 72° C. The amino acid sequence of 36A Tag is SMTGGQQMGRDLYDDDDKDRWGS and its nucleotide sequence is TCC ATG ACC GGA GGT CAG CAG ATG GGT CGC GAC CTG TAC GAC GAT GAC GAC MG GAC AGA TGG GGA AGC. The nucleotide sequence of 36A is inserted as XhoI-36A-Stop-XbaI between MCS and BGH pA. More specific descriptions for 35A Tag is disclosed in Korean Patent No 10-0295558.

FIG. 3 shows the results of Western blotting for liver cancer antigens expressed in transformed cells. MH134 cells were transformed with the recombinant pcDNA3.1-HA-36A/AFP, pcDNA3.1-HA-36A/MAGEA1, pcDNA3.1-HA-36A/TRP53, and pcDNA3.1-HA-36A/GPC3, and selected in the presence of antibiotics G418, followed by Western blotting. As an antibody for analysis, gene-specific monoclonal antibody was used (anti-AFP antibody, H-140 Santacruz; anti-MAGEA1 antibody, ab3211 Abcam; anti-TRP53 antibody, MAB1355 R&D systems; anti-GPC3 antibody, AF2199 R&D systems).

FIG. 4 represents the results of Western blotting showing the expression stability of liver cancer antigens (AFP, P53, MAGEA1 and GPC3) introduced into MI-1134 cell. Cell lines established were cultured in the absence of G418 and 1×10⁶ cells were subjected to Western blotting. Nc denotes a negative control, non-transformed MH134 cells.

FIG. 5 shows the results of SDS-PAGE analysis and Western blotting analysis for liver cancer antigens (AFP, MAGEA1, GPC3, TRP53 and NY-ESO-1). The nucleotide sequences encoding liver cancer antigens were cloned into pCTP vector and expressed in BL21-gold (DE3). The recombinant CTP-conjugated proteins expressed were confirmed by 12% SDS-PAGE and Western blotting. Lanes M, 1, 2, 3, 4, 5, 6, 7, 8, 9, 10, 11, and 12 correspond to molecular weight marker, pellets and supernatant of CTP-AFP 1/2N, pellets and supernatant of CTP-AFP 2/3N, pellets and supernatant of CTP-GPC3 1/2N, pellets and supernatant of CTP-TRP53, pellets and supernatant of CTP-NY-ESO1, pellets and supernatant of CTP-MAGEA1 pellets and supernatant of CTP-MAGEA3, respectively.

FIGS. 6a and 6b represent the relative growth rate of solid cancer in C3H/HeN mice induced by liver cancer antigen-expressing recombinant MH134 and control MH134 cells. 3×10⁵ cells of recombinant MH134 or control MH134 were subcutaneously inoculated into C3H/HeN mice and the formation and rate of cancer were observed after 30-days (FIG. 6a). 5×10⁵ cells of recombinant MH134 or control MH134 were subcutaneously inoculated into C3H/HeN mice and the formation and rate of cancer were observed after 30-days (FIG. 6b). Following the inoculation of recombinant tumor cell lines, the size of tumor was measured in a time interval of 3 days.

FIG. 7 represents the prevention effects of DC (dendritic cell)-based vaccines to inhibit tumorigenesis induced by recombinant MH134 cell lines. For investigating the prevention efficacy of DC pulsed with liver cancer antigens, 1×10⁶ cells/mouse of pulsed DC were subcutaneously injected twice into mice in a time interval of one week. 1-week later, 1×10⁶ cells/mouse of recombinant cancer cell lines were subcutaneously injected into mice. Thereafter, the size of tumor was measured in a time interval of 2 days.

FIG. 8 represents survival rates of mice immunized with DC-based vaccines in cancer prevention model. Mice were immunized with DC vaccines and challenged with recombinant cancer cell lines. The number of mouse which survived was counted. The mouse injected with DC vaccines survived even if all of the control mice died at 50-days.

FIG. 9 shows the prevention efficacy of DC vaccines to inhibit pulmonary metastasis. Mice were administered twice with DC pulsed with CTP-AFP in a time interval of one week. Then, a recombinant liver cancer cell line (MH134/AFP) was inoculated into mice via tail vein. After 20 days of inoculation, lung was extracted and photographed, and the number of cancer nodules formed was counted.

FIG. 10 represents the treatment efficacy of DC vaccine for cancer in mice harboring tumor. 3×10⁵ cells/mouse of recombinant cancer cell lines expressing human liver cancer antigens were subcutaneously injected into mice. 3-day later, 1×10⁶ cells/mouse of bone marrow-derived dendtiric cells (Bm-DC) pulsed with a recombinant liver cancer antigen CTP-MAGEA1 or CTP-AFP were subcutaneously injected twice into mice in a time interval of one week. After 2 days of the injection, the formation and size of tumor were examined every 2 days. On 20 day of injection, the tumor in mice was photographed.

FIG. 11a represents the activities of cancer antigen-specific cytotoxic T lymphocytes in mice treated with DC vaccines. T lymphocytes were isolated from spleen of mice treated with DC vaccines and mixed with antigen presenting cells (APC) pulsed with each CTP-antigen at a ratio of 5:1 (T:APC). Following 5 days of incubation, the activities of cytotoxic T lymphocytes were measured. The expression levels of IFN-γ and IL-4 were examined by ELISA (FIG. 11b), and proliferation abilities of T-cell was investigated with MTT assay (FIG. 11c).

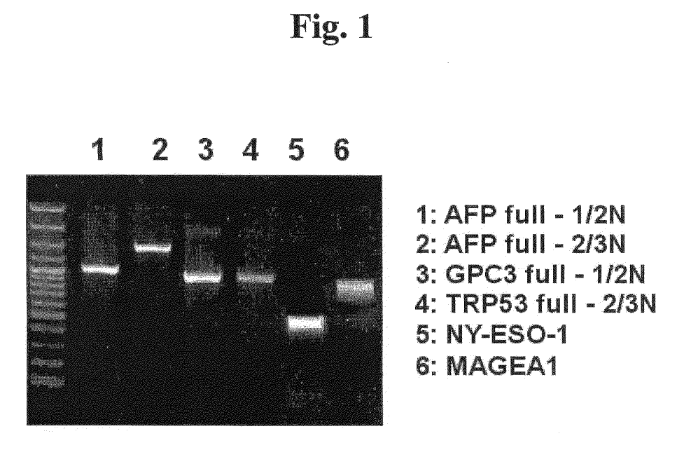

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1

### Preparation of Mouse Cell Lines Expressing Human-Derived Liver Cancer Antigens

### Example 1-1

### Construction of Expression Vectors for Human-Derived Liver Cancer Antigens

### (a) Culture of Human-Derived Liver Cancer Cell Line HepG2, ZR75-1, SK-BR-3.

The HepG2, ZR75-1, SK-BR-3 used in this experiment is human-derived liver cancer cell line expressing human liver cancer-specific antigens such as AFP (Alpha-Fetoprotein), TRP53 (Transformation Related Protein 53), GPC3 (Glypican3), MAGEA1 (Melanoma Antigen Family A, 1), NY-ESO-1 (New York Esophageal Squamous Cell Carcinoma 1 or Cancer/Testis Antigen1; CTAG1) and was obtained from the Korean Cell Line Research Foundation. The liver cancer cell line was cultured and maintained in RPMI-1640 medium (Gibco/BRL) containing 10% FBS. Cultured cells were treated with trypsin-EDTA for 1 min to obtain non-adherent single cells and then subcultured to 80% confluency. The subculturing was carried out 2-3 times a week.

(b) Preparation of cDNA PCR Products of AFP (Alpha-Fetoprotein), GPC3 (Glypican3), MAGEA 1 (Melanoma Antigen Family A, 1) in HepG2, and NY-ESO-1 (New York Esophageal Squamous Cell Carcinoma 1 or Cancer/Testis Antigen1; CTAG1), MAGEA 1 (Melanoma Antigen Family A, 1) in SK-BR-3.

Prior to harvesting liver cancer cell line, cells were subcultured 2-3 times to 60% confluency and trypsinized, followed by harvesting cells. Total RNA was extracted using Trizol (Gibco BRL) from cells harvested and subjected to isopropanol precipitation and 70% ethanol washing for purification. For synthesizing cDNA, a mixture of 10 μg of total RNA and 1 μg of oligo (dT) 12-18 primer were denatured for 5 min at 65° C. and transferred on ice, to which reverse transcriptase buffer, 10 mM DTT, 1 mM dNTP mixture and 20 units RNAsin were added. The reactant mixture was prereacted for 2 min at 42° C. and then underwent reverse transcription using 200 units MMLV (Molony Murine Leukemia Virus) reverse transcriptase (Invitrogen, Inc.) for 60 min at 42° C. After the completion of reactions, the reactions were kept to stand for 15 min at 70° C. to inactivate the enzyme. PCR reactions were carried out using cDNA molecules synthesized as templates for amplifying cDNA molecules of AFP (Alpha-Fetoprotein), MAGEA 1 (Melanoma Antigen Family A, 1), GPC3 (Glypican3), TRP53 (Transformation Related Protein 53), and NY-ESO-1 (New York Esophageal Squamous Cell Carcinoma 1 or Cancer/Testis Antigen1; CTAG1). The primer sequences used are summarized in Tables 1a and 1b.

TABLE 1a
Primers for Cloning into Prokaryotic Expression Vectors

| | | |
|---|---|---|
| Target gene | Primer | Sequence |

| | | |
|---|---|---|
| hAFP | hAFP-partial-F | 5′-GGGGTACCACACTGCATAGAAATGAATATGGAAT-3′ |
| | hAFP-partial-R | 5′-CGGAATTCTTAAACTCCCAAAGCAGCACGA-3′ |
| | hAFP-partial-R-1/2 | 5′-CGGAATTCTTATTCCCCTGAAGAAAATTGG-3′ |
| | hAFP-partial-R-2/3 | 5′-CGGAATTCTTATAAGTGTCCGATAATAATGTCAGC-3′ |
| hGPC3 | hGPC3-partial-F | 5′-GGGGTACCCCGGACGCCACCTGTCAC-3′ |
| | hGPC3-partial-R | 5′-CGGAATTCTCAGTGCACCAGGAAGAAGAAGC-3′ |
| | hGPC3-partial-R-1/2 | 5′-CGGAATTCTCACTGGATAGAATCATGGATTGTTG-3′ |
| hTRP53 | hTP53-partial-F | 5′-GGGGTACCGAGGAGCCGCAGTCAGATC-3′ |
| | hTP53-partial-R | 5′-CGGAATTCTCAGTCTGAGTCAGGCCCTTCT-3′ |
| | hTP53-partial-R-2/3 | 5′-CGGAATTCTCATTCTCCATCCAGTGGTTTCTTC-3′ |
| hCTAG1(NY- | hCTAG1-partial-F | 5′-GGGGTACCCAGGCCGAAGGCCGGGGCA-3′ |
| ESO-1) | hCTAG1-partial-R | 5′-CGGAATTCTTAGCGCCTCTGCCCTGAGGGAGGCTG-3′ |
| hMAGEA-1 | hMAGE1-partial-F | 5′-AGGGGTACCTCTCTTGAGCAGAGGAGTCT-3′ |
| | hMAGE1-partial-R | 5′-AGGGAATTCTCAGACTCCCTCTTCCTCCT-3′ |

TABLE 1b
Primers for Cloning into Eukaryotic Expression Vectors

| | | |
|---|---|---|
| Target gene | Primer | Sequence |

| | | |
|---|---|---|
| hAFP | hAFP-full-F | 5′-GGGGTACCATGAAGTGGGTGGAATCAATTT-3′ |
| | hAFP-full-R | 5′-CGGAATTCCCAACTCCCAAAGCAGCACGA-3′ |
| | hAFP-full-R-1/2N | 5′-CGGAATTCCCTTCCCCTGAAGAAAATTGG-3′ |
| | hAFP-full-R-2/3N | 5′-CGGAATTCCCTAAGTGTCCGATAATAATGTCAGC-3′ |
| hGPC3 | hGPC3-full-F | 5′-GGGGTACCATGGCCGGGACCGTGCGC-3′ |
| | hGPC3-full-R | 5′-CGGAATTCCCGTGCACCAGGAAGAAGAAGC-3′ |
| | hGPC3-full-R-1/2N | 5′-CGGAATTCCCCTGGATAGAATCATGGATTGTTG-3′ |
| hTRP53 | hTP53-full-F | 5′-GGGGTACCATGGAGGAGCCGCAGTCAGA-3′ |
| | hTP53-full-R | 5′-CGGAATTCCCGTCTGAGTCAGGCCCTTCTGT-3′ |
| | hTP53-full-R-2/3N | 5′-CGGAATTCCCTTCTCCATCCAGTGGTTTCTTC-3′ |
| hCTAG1(NY- | hCTAG1-full-F | 5′-GGGGTACCATGCAGGCCGAAGGCCGGGGCA-3′ |
| ESO-1) | hCTAG1-full-R | 5′-CGGAATTCCCGCGCCTCTGCCCTGAGGGAGGCTG-3′ |
| MAGE-1 | hMAGE1-full-F | 5′-AGGGGTACCATGTCTCTTGAGCAGAGGAG-3′ |
| | hMAGE1-full-R | 5′-AGGGAATTCCCGACTCCCTCTTCCTCCTC-3′ |

Using primer sets (Bionics, Inc.) in Table 1a and PCR polymerase (Solgent Co., Ltd.), PCR amplifications were conducted for obtaining DNA fragments, GPC3 (Glypican3) (909 bp), TRP53 (Transformation Related Protein 53) (978 bp), NY-ESO-1 (New York Esophageal Squamous Cell Carcinoma 1 OR Cancer/Testis Antigen1; CTAG1) (540 bp), AFP (Alpha-Fetoprotein) (983 bp) and MAGEA1 (Melanoma Antigen Family A, 1) (927 bp), for expressing in prokaryotic cells under the following thermal conditions: 25 cycles of 30 sec at 94° C., 30 sec at 62° C., and 50 sec at 72° C. Likely, for obtaining DNA fragments, GPC3 (Glypican3) (998 bp), TRP53 (Transformation Related Protein 53) (980 bp), NY-ESO-1 (New York Esophageal Squamous Cell Carcinoma 1 OR Cancer/Testis Antigen1; CTAG1) (542 bp), AFP (Alpha-Fetoprotein) (1040 bp) and MAGEA1 (Melanoma Antigen Family A, 1) (929 bp), for expressing in eukaryotic cells, PCR amplifications were conducted using primer sets in Table 1b. For making it feasible to detect proteins expressed in eukaryotic cells, 36A Tag sequence developed by CreaGen, Inc. (Korea) was introduced to the amplified nucleotide sequences. Primers for introducing Tag sequence are Tag-XhoI/s(5′-ACCCTCGAGGTCCATGACCGGAGGTCAGC AGATGGGTCGCGACCTGTACGACGA-3′) and Tag-XbaI/as (5′-ACCTCTAGATTAGCTICCCCATCTGTCCTTGTCGTCATCGTCGTACAGGTCGCG-S′). Tag DNA fragments were prepared by PCR amplification under the thermal conditions: 1 cycle of 30 sec at 94° C., 30 sec at 52° C., and 5 min at 72° C.

The amino acid sequence of 36A Tag is SMTGGQQMGRDLYDDDDKDRWGS and its nucleotide sequence is TCC ATG ACC GGA GGT CAG CAG ATG GGT CGC GAC CTG TAC GAC GAT GAC GAC AAG GAC AGA TGG GGA AGC. The nucleotide sequence of 36A is inserted as XhoI-36A-Stop-XbaI between MCS and BGH pA. More specific descriptions for 35A Tag is disclosed in Korean Patent No 10-0295558.

(c) Cloning Liver Cancer Antigen cDNA into Expression Vectors (pcDNA3.1(+)-36A Tag Vector and pCTP Vector)

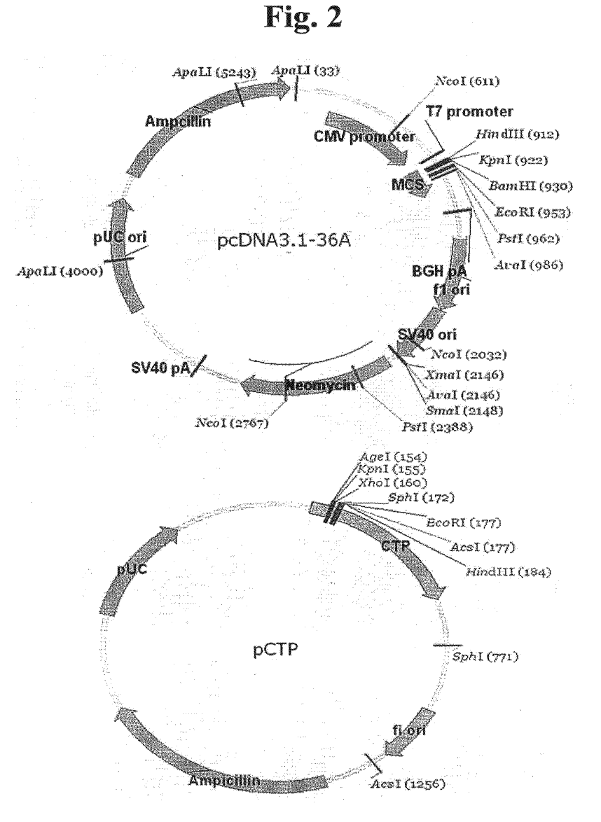

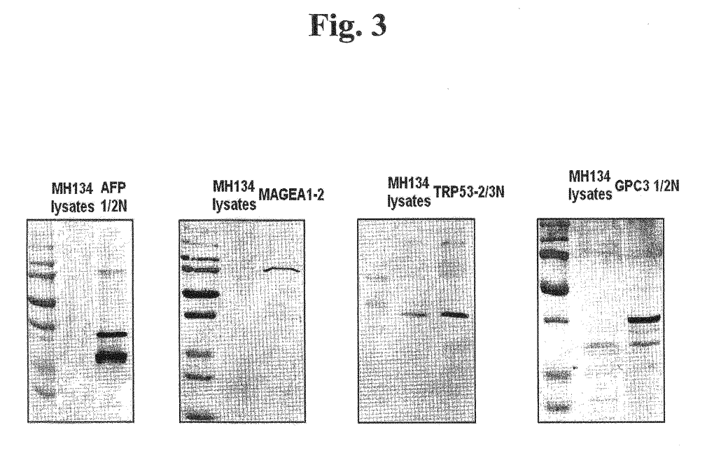

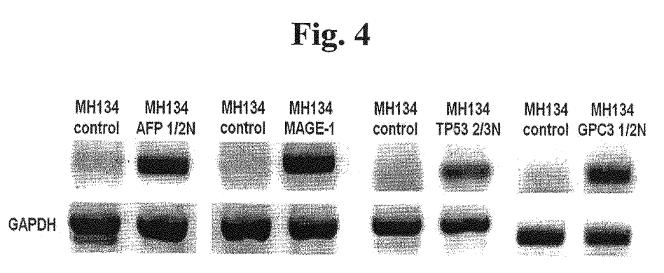

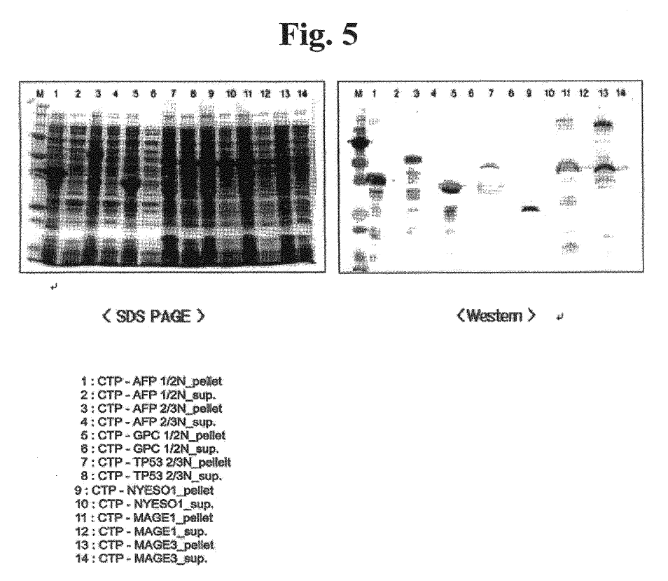

Each of DNA fragments for human liver cancer antigens was digested using KpnI/EcoRI and cloned into pcDNA3.1(+)-Tag vector, followed by confirming cloned sequences by sequencing (see FIG. 2 and SEQ ID NO: 1-SEQ ID NO: 6). To obtain recombinant liver cancer antigens in prokaryotic cells, pCTP-Td vector was used. pCTP-Td vector was constructed by genetically manipulating pTAT-HA vector (kindly provided by Dr. S. Dowdy at the Washington University, H. Nagahara et al., Nature Med. 4:1449 (1998)). Each of DNA fragments for human liver cancer antigens was digested using KpnI/EcoRI and cloned into pCTP vector, followed by confirming cloned sequences by sequencing (see FIG. 2 and SEQ ID NO: 7-SEQ ID NO: 12).

DNA sequencing was undertaken for the nucleotides sequences cloned. It was verified that the amino acid sequences encoded by the cloned sequences had 100% identity to known amino acid sequences of AFP, MAGEA1, GPC3, TRP53 and NY-ESO-1 (Blast 2 sequence search).

The sequences introduced into prokaryotic expression vectors are lack of sequences corresponding to N-terminal signal peptide and transmembrane domain adjacent to C-terminal. The nucleotide sequences into prokaryotic expression vectors encode amino acids 20-346 of AFP (327 aa) (SEQ ID NO: 19), amino acids 31-331 of GPC3 (303 aa) (SEQ ID NO: 21), amino acids 1-326 of TRP53 (326 aa) (SEQ ID NO: 22), amino acids 1-180 of NY-ESO-1 (180 aa) (SEQ ID NO: 23) and amino acids 1-308 of MAGEA1 (308 aa) (SEQ ID NO: 24). In the meantime, the nucleotide sequences into eukaryotic expression vectors encode amino acids 1-346 of AFP (346 aa) (SEQ ID NO: 13), amino acids 1-332 of GPC3 (332 aa) (SEQ ID NO: 15), amino acids 1-326 of TRP53 (326 aa) (SEQ ID NO: 16), amino acids 1-180 of NY-ESO-1 (180 aa) (SEQ ID NO: 17) and amino acids 1-309 of MAGEA1 (309 aa) (SEQ ID NO: 18).

### Example 1-2

### Establishment of Human-Derived Liver Cancer Antigen-Expressing Mouse Cell Lines

### (a) Analysis of Antigen Expression in Liver Cancer Antigen-Expressing Mouse Cell Lines

To prepare liver cancer antigen-expressing cell lines, pcDNA3.1(+)-Tag/liver cancer antigen (AFP, SEQ ID NO: 1; GPC3, SEQ ID NO: 3; TRP53, SEQ ID: 4; MAGEA1, SEQ ID NO: 6) vectors were transformed into mouse liver cancer cell line MH134 cells.

The constructs cloned in 20 μg of eukaryotic cell expression vector pcDNA3.1 (+)-36A were linearized by the treatment of restriction enzyme (Ssp I; Pvu I for AFP) at 37° C. for 2 hr. Enzyme treated pcDNA3.1 was purified using PCR purification kit. 2×10⁵ cells of MH134 cell line were mixed with 50 μl of the final eluted DNA in 550 μl of 1×PBS by resuspending, and then 2 μl of 2 M MgCl₂ (final concentration 5 mM) was added. The final 660 μl of DNA and MH134 cell mixture was put into electroporation cuvette and stayed in the ice. After that, electroporation was exerted using BIO-RAD gene pulser at 280V, 950 μF, and then, incubated for 10 min in ice. The mixture of DNA and MH134 cell in the cuvette was transferred to 50 ml tube containing 10 ml of RPMI1640 and 10% FBS by using yellow tip. The mixture was divided into 96 well microplate with 100 μl per well. After incubating for 2 days at 37° C., G418 (10 mg/ml) was added into the well to make the final concentration of G418 be 1 mg/ml. After treatment of G418, the formation of cell colony was observed. The well having cell colony was selected, transferred to 6 well plate, and after that, it is transferred to 100 mm dish when cells were grown at the concentration of 10⁶ cells/ml. Cells selected were proliferated and harvested and in turn the expression pattern of antigens was verified by Western blotting analysis. Harvested cells were washed twice with PBS, heated in protein sample buffer and centrifuged to remove genomic DNA molecules, followed by SDS-PAGE for supernatant isolation. Protein bands resolved were transferred to a nitrocellulose membrane using semi-dry transfer blotter (Bio-Rad) and incubated with a primary antibody, Tag antigen-specific monoclonal antibody and a secondary antibody AP (alkaline phosphatase)-conjugated anti-mouse IgG (Sigma). The bands were visualized using AP reaction solution (Promega) containing NBT/BCIP.

### (b) Evaluation of Cell Line Stability in View of Antigen Expression

To verify whether recombinant cell lines established maintain the antigen expression potential in the absence of antibiotics (G418) when injected into mice, cell lines were subcultured in a medium with no G418 and examined, so that the stable expression of introduced foreign sequences were checked. Each of cell lines (MH134/AFP, MH134/GPC3, MH134/TRP53 and MH134/MAGEA1) was cultured in the absence of G418, 1×10⁶ cells were harvested every three days and then their antigen expression potential was examined by RT-PCR using primer specific for each antigen.

TABLE 1c

Primers for identifying the expression
of eukaryotic expression vector and
primer sequence for target gene.

| | | | | | | | | |
|---|---|---|---|---|---|---|---|---|
| OLIGO | start | Len | tm | Gc% | any | 3′ | rep | Sequence |

MAGEA-1

| | | | | | | | | |
|---|---|---|---|---|---|---|---|---|
| LEFT | 150 | 20 | 60.05 | 55.00 | 4.00 | 0.00 | 11.00 | GTCAACAGATCCTCCCCAGA |
| PRIMER | | | | | | | | |
| RIGHT | 387 | 20 | 59.99 | 45.00 | 5.00 | 1.00 | 12.00 | CAGCATTTCTGCCTTTGTGA |
| PRIMER | | | | | | | | |

SEQUENCE SIZE: 930
INCLUDED REGION SIZE: 930
PRODUCT SIZE: 238
AFP 1/2N

| | | | | | | | | |
|---|---|---|---|---|---|---|---|---|
| LEFT | 381 | 20 | 60.15 | 50.00 | 2.00 | 2.00 | 10.00 | ACACAAAAAGCCCACTCCAG |
| PRIMER | | | | | | | | |
| RIGHT | 595 | 20 | 59.75 | 45.00 | 5.00 | 2.00 | 11.00 | CTGCATTTTCAGCTTTGCAG |
| PRIMER | | | | | | | | |

SEQUENCE SIZE: 900
INCLUDED REGION SIZE: 900
PRODUCT SIZE: 215
TRP53 (TRANSFORMATION RELATED PROTEIN 53) 2/3N

| | | | | | | | | |
|---|---|---|---|---|---|---|---|---|
| LEFT | 35 | 20 | 60.23 | 55.00 | 7.00 | 2.00 | 12.00 | CCCCTCTGAGTCAGGAAACA |
| PRIMER | | | | | | | | |
| RIGHT | 185 | 20 | 60.05 | 55.00 | 6.00 | 0.00 | 11.00 | TCATCTGGACCTGGGTCTTC |
| PRIMER | | | | | | | | |

SEQUENCE SIZE: 550
INCLUDED REGION SIZE: 550
PRODUCT SIZE: 151
GPC3 (GLYPICAN3) 1/2N

| | | | | | | | | |
|---|---|---|---|---|---|---|---|---|
| LEFT | 562 | 20 | 60.07 | 50.00 | 7.00 | 2.00 | 10.00 | CCTGATTCAGCCTTGGACAT |
| PRIMER | | | | | | | | |
| RIGHT | 801 | 20 | 60.01 | 55.00 | 5.00 | 1.00 | 10.00 | TCCCTGGCAGTAAGAGCAGT |
| PRIMER | | | | | | | | |

SEQUENCE SIZE: 871
INCLUDED REGION SIZE: 871
PRODUCT SIZE: 240

### Example 2

### Purification of Recombinant CTP-Conjugated Proteins for Pulsing Dendritic Cells and Measurement of Transduction Potential

### Example 2-1

### Expression and Purification of Recombinant CTP-Conjugated Liver Cancer Antigens

E. coli BL21Gold (DE3) competent cells (Stratagene) were transformed with recombinant pCTP-Td vectors carrying cDNA for each liver cancer antigen (see SEQ ID NO:7 and SEQ ID NO:12) to prepare transformants according to Hanahan method and cultured in ampicillin-LB medium. The transformants cultured were centrifuged, washed with PBS and harvested, followed by analyzing liver cancer antigen expression on 12% SDS-PAGE. Following the expression, recombinant proteins CTP-AFP, CTP-MAGEA1, CTP-TRP53, CTP-GPC3, and CTP-NY-ESO-1 were purified through a column of Ni⁺-NTA resin(Qiagen). The proteins analyzed were shown to have a molecular weight higher by about 6 kDa, which is ascribed to non-genome sequence originated from vectors. In other words, CTP-AFP shows a molecular weight of about 44 kDa, CTP-MAGEA1 of about 48 kDa, CTP-GPC3 of about 41 kDa, CTP-TRP53 of about 53 kDa and CTP-NY-ESO-1 of about 30 kDa.

### Example 3

### Establishment of Animal Liver Cancer Models

### Example 3-1

### Evaluation of Formation and Growth of Cancer Caused by Liver Cancer Antigen-Expressing Cell Lines in Mice

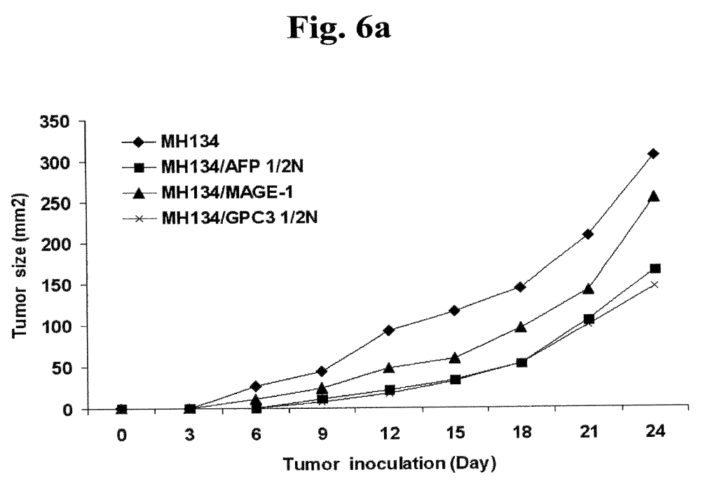

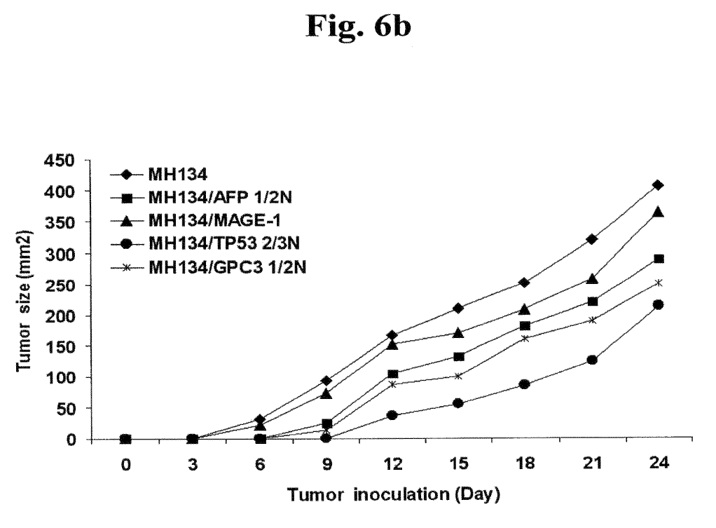

We examined the formation and growth of cancer caused by mouse cell lines expressing human liver cancer antigens. Cell lines prepared hereinabove were injected into femurs of 6-week-old Balb/c mouse (Orient, Inc., Korea). The recombinant cell lines expressing human liver cancer antigens were cultured and maintained in RPMI medium containing 10% FBS and 500 μg/ml G418. Cells at optimal growth state were washed 2-3 times with PBS, treated with trypsin-EDTA for single cell isolation, and suspended at 3×10⁵ cells/100 μl and 5×10⁵ cells/100 μl in PBS. 100 μl of the suspension were subcutaneously inoculated into mice. Following the inoculation of cell lines, the formation of solid cancer was observed in a time interval of 3 days. The size of solid cancer was measured using calipers. As represented in FIG. 6b, all mice inoculated with liver cancer cell lines were found to bear solid cancer. Interestingly, a lower dose of cells, e.g. 3×10⁵ cells could cause tumorigeneisis and cancer formed in mice were not extinguished by immune response to heterologous antigens. FIG. 6b shows the growth rate of cancer. The recombinant MH134/TRP53 cell line shows a growth rate of cancer lower than other cell lines and the MH134/MAGEA1 cell shows a growth rate of cancer similar to MH134. It would be understood that the expression of TRP53 is made in the highest level and in turn elicits the strongest immune responses. These results demonstrate that the tumorigenesis in mice caused by cells expressing human liver cancer antigens could not be prevented by immune responses to heterologous antigens. Accordingly, it could be determined that liver cancer mouse models are successfully established by the present invention and allows for evaluating the prevention and treatment efficacy of dendritic cell-based vaccines.

### Example 4

### Analysis of Anti-Cancer Efficacy of Dendritic Cells

### Example 4-1

### Prevention Efficacy of Dendritic Cell-Based Vaccine (Prevention Model)

To investigate whether dendritic cell-based vaccines can prevent liver cancer, mice were immunized twice with dendritic cells pulsed with recombinant CTP-conjugated liver cancer antigens and challenged with cancer cell lines expressing liver cancer-specific antigen, followed by examining the formation of solid cancer and pulmonary metastasis.

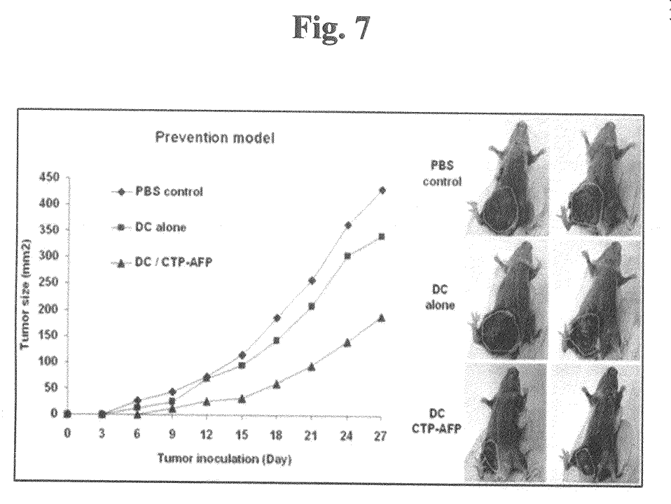

Mice dendritic cells were prepared by differentiating bone marrow cells of a femur and tibia into dendritic cells. The both ends of a femur and tibia were dissected, from which bone marrow cells were extracted and collected into a 50 ml tube. Bone marrow cells collected were suspended in 0.83% ammonium chloride solution to remove red blood cells, and cultured in a dendritic cell production medium (RPMI-1640 medium containing 10% FBS, 10 ng/ml mouse recombinant IL-4 and 10 ng/ml mouse GM-CSF) for 2 days. Non-adherent cells were removed to obtain only adherent cells on the bottom of tubes. Medium was changed with a fresh medium in a time interval of 2-3 days to prevent the deficiency of cytokines. On a 6 day of culture, immature dendritic cells were harvested and incubated with CTP-AFP. Immature dendritic cells were pulsed with 50 μg/ml of each antigen protein for 20 hr, to which 100 μg/ml of IFN-γ and 100 μg/ml of TNF-α as cytokines for DC maturation were added. 1×10⁶ cells of dendritic cells pulsed with antigens were subcutaneously injected into mice to elicit immune reactions to cancer. Immunization with dendritic cells was conducted twice in a time interval of 2 weeks. After one week of the second immunization, mice immunized by dendritic cells pulsed with CTP-AFP were subcutaneously challenged with 3×10⁵ cells/mouse of MH134/AFP. The size of cancer (length x breadth) was measured every three days. As shown in FIG. 7, mice immunized using dendritic cells pulsed with CTP-AFP antigen were shown to bear no tumor mass.

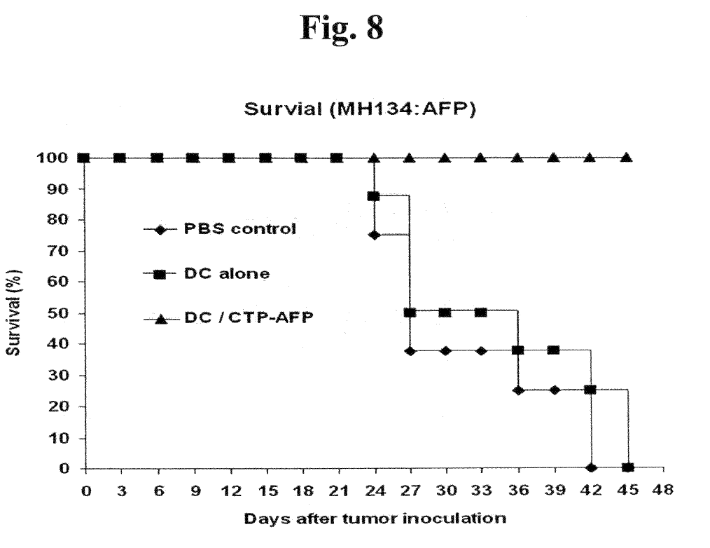

FIG. 8 graphically shows the cancer incidence in a cancer prevention model using dendritic cells. In the group of mice immunized with CTP-AFP pulsed dendritic cells, mice challenged with MH134/AFP cell lines were found to exhibit retardation of the tumorigenesis and survived after 48 days. On the contrary, in the control group of mice treated with PBS, mice started to die at 25 days and all of mice died at 42 days. In the control group of mice immunized with non-pulsed dendritic cells, mice started to die at 42 days and all of mice died at 45 days. Cancer prevention model using dendritic cells pulsed with cancer specific antigen exhibits the efficacy of life extension as well as retardation of tumorigenesis.

### Example 4-2

### Inhibition of Liver Cancer Metastasis by Dendritic Cell-Based Vaccine (Prevention Model)

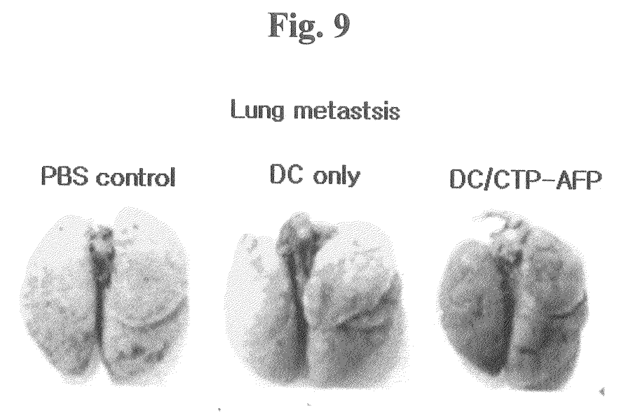

Mice were immunized twice with dendritic cell-based vaccines as described hereinabove and injected via tail vein with 3×10⁵ cells/mouse of cell lines expressing liver cancer antigens. MH134 mouse liver cancer cell lines exhibit spontaneous metastasis. 20 days later, mice were euthanized and pulmonary metastasis was evaluated. As shown in FIG. 9, mice immunized using dendritic cells pulsed with human liver cancer antigen AFP were shown to exhibit no pulmonary metastasis. In contrast, mice treated with either dendritic cells not pulsed or PBS were shown to elicit strong pulmonary metastasis. It could be understood that dendritic cell-based vaccines elicit strong immune reactions specific to cancer antigens and in turn inhibit the formation and metastasis of cancer.

### Example 4-3

### Treatment Efficacy of Dendritic Cell-Based Vaccine (Regression Model)

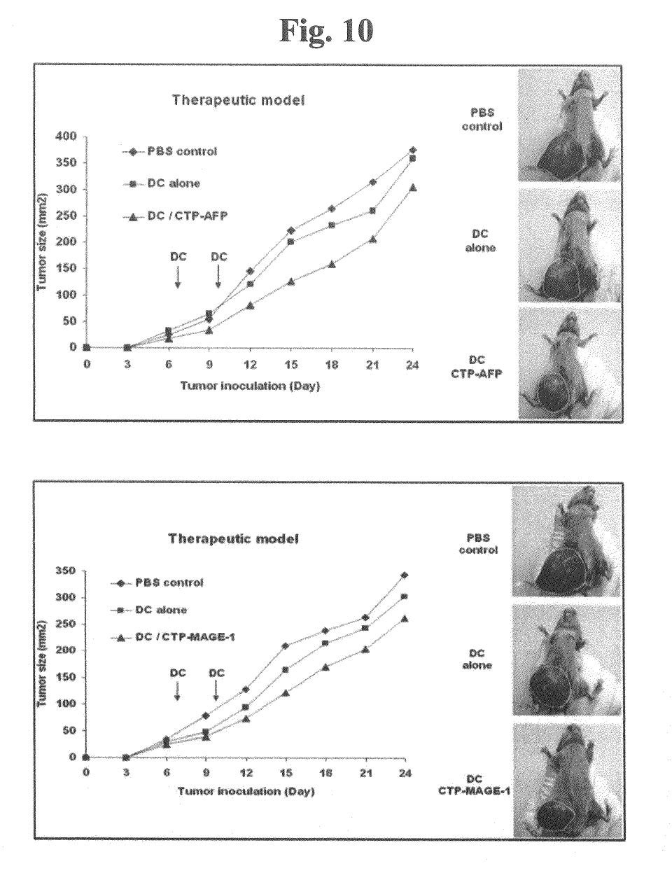

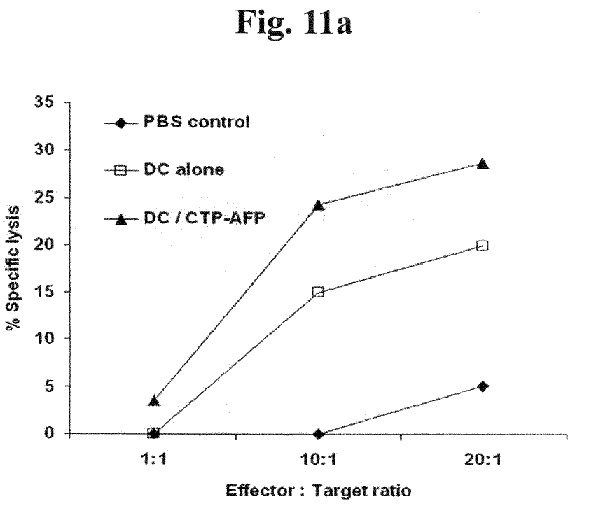

Mice were subcutaneously injected with 3×10⁵ cells/mouse of recombinant cell lines expressing liver cancer antigen AFP. After 3 days, mice were injected twice in a time interval of one week with 1×10⁶ cells/mouse of dendritic cells pulsed with CTP-conjugated antigens (CTP-MAGEA1 and CTP-AFP). Following the second administration of DC, the formation and growth of cancer were observed for one month in a time interval of 2 days. As represented in FIG. 10, the growth of cancer was inhibited in all of liver cancer mouse models established using MH134/MAGEA1 and MH134/AFP cell lines.

### Example 4-4

### Analysis of CTL Response Induced by Dendritic Cell-Based Vaccine

T cell proliferation and CTL activity were analyzed using splenocytes isolated from mice of pulmonary metastasis model. Each mouse was euthanized according to cervical dislocation method and spleen was isolated and stored in RPMI. Each spleen was passed through a 70 μm sieve and suspended tissues were then removed. The resultant was centrifuged to collect cells, after which were suspended in 0.83% ammonium chloride solution to remove red blood cells. The splenocytes prepared were passed through a nylon wool column to isolate T lymphocytes as effector cells, and mixed with APC (antigen presenting cells) at a ration of 5:1, followed by culturing for 5 days. APC was prepared 2 days prior to experiment. Separately, splenocytes were isolated from normal mice and treated for 24 hr with 3 μg/ml of Con-A. The stimulated cells were incubated with 50 μg/ml of each antigen protein CTP-AFP for 24 hr. The cell concentration was maintained at 1×10⁶ cells during culture. After 24-hr culture, cells were treated with mitomycin C for 40 min and washed three times to prepare APC. After 3-days culture of effector T cell and APC, T cell proliferation assay (MTT assay) was performed as to a portion of culture. After 4-days of the culture, the amount of IL-4 and IFN-γ in the supernatant was measured. R&D systems kit was used according to the indication of the manufacturer. Specific lysis of MH134 cell was detected by CFSE staining method since the MH134 is suspension cell. As non-target cells MH134 cells were used, and as target cells stabilized cell line expressing antigen were used. In order to differentiate the non-target cell line from the target cell line, cells are stained by CFSE after washed. (Target: add 15 μl of 1 mM CFSE (CFSE high) vs Non target cell: add 10 μl of 0.1 mM CFSE (CFSE low)). After mixing two kinds of stained cell lines with identical ratio, effector T cells were isolated by removing dead cells with Histopaque (Sigma). The effector T cell and target cell were mixed at Er ratios of 1:1, 10:1, and 20:1 and incubated for 6 hr. After that, the number of live cells was measured through FACS analysis.

| E:T ratio | Effector cells | Target mix | RPMI-10 |
|---|---|---|---|
| 0.5:1 | 25 μl | 100 μl | 175 μl |
| 1:1 | 50 μl | 100 μl | 150 μl |
| 2:1 | 100 μl | 100 μl | 100 μl |
| 4:1 | 200 μl | 100 μl | |
| Target only | | 100 μl | 200 μl |

After that, values were calculated by the equation below.

Percent of specific lysis=(1−the ratio of target cells only/the ratio of target+effector cells)×100.

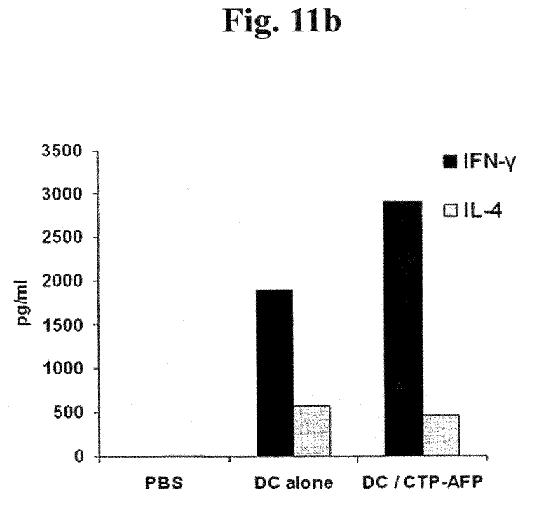

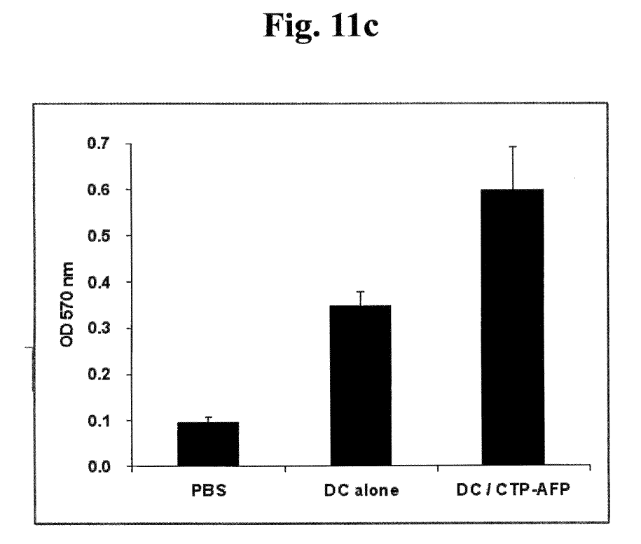

As represented in FIG. 11, CTLs were effectively induced in all three mouse models. These results demonstrate that the administration of dendritic cell-based vaccines pulsed with CTP-AFP induces effectively CTLs specific to human liver cancer antigens, giving rise to the prevention and treatment efficacy to cancer.

As described previously, the present invention provides methods for analyzing the prevention and treatment efficacy of dendritic cells as immunotherapeutics for liver cancer by use of animal models. For executing the prevention or treatment of liver cancer using dendritic cells in a clinical scale, it is prerequisite to verity the efficacy and safety of dendritic cells in animal models. The present invention allows for animal model-based evaluation of dendritic cells as immunotherapeutics. Dendritic cell-based vaccines (DC vaccines) selected by the present invention become promising candidates as immunotherapeutics for liver cancer.

Having described a preferred embodiment of the present invention, it is to be understood that variants and modifications thereof falling within the spirit of the invention may become apparent to those skilled in this art, and the scope of this invention is to be determined by appended claims and their equivalents.

1. A method for analyzing the prevention and treatment efficacy of a dendritic cell-derived immunotherapeutic for liver cancer using an animal model carrying tumors expressing a human liver cancer-specific antigen, which comprises the steps of:
(a) administering to a normal animal other than human a cancer cell line expressing the human liver cancer-specific antigen to induce cancer in the normal animal;
(b) administering to the animal with cancer dendritic cells to be analyzed; and
(c) determining the prevention and treatment efficacy of the dendritic cells as immunotherapeutics for cancer by measuring the formation or growth of cancer cells originated from the cancer cell line in the animal.

2. A method for analyzing the prevention and treatment efficacy of a dendritic cell-derived immunotherapeutic for liver cancer using an animal model carrying tumors expressing a human liver cancer-specific antigen, which comprises the steps of:
(a) administering to a normal animal other than human dendritic cells to be analyzed;
(b) administering to the animal the cancer cell line expressing the human liver cancer-specific antigen to induce cancer in the animal; and
(c) determining the prevention and treatment efficacy of the dendritic cells as immunotherapeutics for liver cancer by measuring the formation or growth of cancer cells originated from the cancer cell line in the animal.

3. The method according to claim 1, wherein the animal is a rodent.

4. The method according to claim 3, wherein the rodent is a mouse (Mus musculus).

5. The method according to claim 1, wherein the human liver cancer-specific antigen is AFP (Alpha-Fetoprotein), MAGEA1 (Melanoma Antigen Family A, 1), TRP53 (Transformation Related Protein 53), GPC3 (Glypican3) or NY-ESO-1 (New York Esophageal Squamous Cell Carcinoma 1 or Cancer/Testis Antigen 1; CTAG1).

6. The method according to claim 5, wherein the human liver cancer-specific antigen is AFP (Alpha-Fetoprotein).

7. The method according to claim 1, wherein the cancer cell line is derived from a mouse (Mus musculus).

8. The method according to claim 7, wherein the cancer cell line is syngeneic to the animal.

9. The method according to claim 1, wherein the administration of dendritic cells or the cancer cell line in the step (a) or (b) is carried out by subcutaneous injection.

10. The method according to claim 1, wherein the administration of dendritic cells or the cancer cell line in the step (b) or (a) is carried out by subcutaneous injection.

11. The method according to claim 1, wherein the cancer cell line expressing the human liver cancer-specific antigen is a liver cancer cell-derived one.

12. A mouse-derived liver cancer cell line (recombinant MH134 cell line) expressing a human liver cancer-specific antigen, characterized in that the human liver cancer-specific antigen is AFP (Alpha-Fetoprotein), MAGEA1 (Melanoma Antigen Family A, 1), TRP53 (Transformation Related Protein 53), GPC3 (Glypican3) or NY-ESO-1 (New York Esophageal Squamous Cell Carcinoma 1 or Cancer/Testis Antigen1; CTAG1).

13. The mouse-derived liver cancer cell line according to claim 12, wherein the cancer cell line is transformed with a vector containing a nucleotide sequence encoding an amino acid sequence of SEQ ID NO: 13, an amino acid sequence of SEQ ID NO: 15, an amino acid sequence of SEQ ID NO: 16, or an amino acid sequence of SEQ ID NO: 18.

14. The mouse-derived liver cancer cell line according to claim 12, wherein the cancer cell line is transformed with a vector containing a nucleotide sequence of nucleotides 7-1044 of SEQ ID NO: 1, a nucleotide sequence of nucleotides 7-1002 of SEQ ID NO: 3, a nucleotide sequence of nucleotides 7-984 of SEQ ID NO: 4, or a nucleotide sequence of nucleotides 7-933 of SEQ ID NO: 6.

15. The mouse-derived liver cancer cell line according to claim 14, wherein the cancer cell line is transformed with pcDNA3.1(+)-Tag/AFP (Alpha-Fetoprotein), pcDNA3.1(+)-Tag/GPC3 (Glypican3), pcDNA3.1(+)-Tag/TRP53 (Transformation Related Protein 53), pcDNA3.1(+)-Tag/NY-ESO-1 (New York Esophageal Squamous Cell Carcinoma 1 or Cancer/Testis Antigen1; CTAG1), or pcDNA3.1(+)-Tag/MAGEA1 (Melanoma Antigen Family A, 1).

16. The mouse-derived liver cancer cell line according to claim 12, wherein the cancer cell line is MH134/AFP (Alpha-Fetoprotein) expressing the AFP (Alpha-Fetoprotein) antigen.

17. A mouse liver cancer model, characterized in that the mouse model has a cancer formed by inoculating the liver cancer cell line of claim 12 expressing the human liver cancer-specific antigen, and the metastasis or growth of the cancer formed in the mouse model is inhibited by the treatment of dendritic cells pulsed with the human liver cancer-specific antigen.

18. The mouse liver cancer model according to claim 17, wherein the liver cancer cell line is syngeneic to the mouse.

19. The mouse liver cancer model according to claim 18, wherein the mouse liver cancer model is used for performing a method for analyzing the prevention and treatment efficacy of a dendritic cell-derived immunotherapeutic for liver cancer using an animal model carrying tumors expressing a human liver cancer-specific antigen, which comprises the steps of:
(a) administering to a normal animal other than human a cancer cell line expressing the human liver cancer-specific antigen to induce cancer in the normal animal;
(b) administering to the animal with cancer dendritic cells to be analyzed; and
(c) determining the prevention and treatment efficacy of the dendritic cells as immunotherapeutics for cancer by measuring the formation or growth of cancer cells originated from the cancer cell line in the animal.
